(19) United States
(12) Patent Application Publication
(10) Pub. No.: US 2023/0116523 A1
(43) Pub. Date: Apr. 13, 2023

(54) NEUROLOGICAL ELECTRODE SYSTEM FOR SENSITIVE SKIN

(71) Applicant: Rhythmlink International, LLC, Columbia, SC (US)

(72) Inventors: Harrison M. Floyd, Columbia, SC (US); Daniel McCoy, Murrells Inlet, SC (US); Shawn Regan, Charlotte, NC (US); Brian Petree, West Columbia, SC (US)

(21) Appl. No.: 18/080,358

(22) Filed: Dec. 13, 2022

Related U.S. Application Data
(63) Continuation-in-part of application No. 16/639,281, filed on Feb. 14, 2020.
(60) Provisional application No. 62/546,678, filed on Aug. 17, 2017.

(51) Int. Cl. A61B 5/291; A61B 5/259

(52) U.S. Cl. CPC A61B 5/291; A61B 5/259; A61B 2503/045; A61B 2562/0215; A61B 2562/0217

(57) ABSTRACT

An electrode system for neurological monitoring is disclosed, including a housing with a recess dimensioned to hold an electrode. A conductive gel is applied to said electrode to limit direct contact by said electrode to a patient's skin. The recess defines a wall in said housing that engages the patient's skin. A thin bead of adhesive applied to the top surface of the wall secures the housing, and therefore the electrode, to the patient. Finer electrical conductor wires in a more flexible insulator lead from the electrode to a connector that fits into a neurological monitoring amplifier. The electrode system further comprising a template structure for precisely locating and adjusting to a patient's dimensions.

### CROSS-REFERENCE TO RELATED APPLICATIONS

This application is a continuation-in-part of, and claims the benefit and priority to, U.S. patent application Ser. No. 16/639,281, filed on Feb. 14, 2020, entitled “A Neurological Electrode System for Sensitive Skin.” Which further claims the benefit of and priority to International Application No. PCT/US2018/000327, having an international filing date of Aug. 17, 2018, which further claims the benefit of and priority to U.S. Provisional Patent Application No. 62/546,678, filed on Aug. 17, 2017, each of which is incorporated herein by reference in their entirety.

### TECHNOLOGICAL FIELD

The present disclosure relates generally to neurological electrodes. In particular, to neurological electrodes and applications thereof for sensitive skin.

### BACKGROUND

Neurological electrodes are used for monitoring and stimulating brain function. For example, when attached to the skin of the scalp of a patient, they can sense nerve impulses inside the brain. However, these electrodes may irritate the skin if they remain attached for a protracted time, and, eventually, cause the skin to breakdown, particularly where the stress of the electrode on the scalp is concentrated, for example, where the rim of a cup-type electrode contacts the skin.

For most patients and most types of neurological monitoring, the effect of the electrodes on the patient's skin is not a significant concern. In caring for newborn babies and others whose skin is sensitive, skin damage is of greater concern. Consequently, there is a need for improved neurological electrode systems and methods for applying multiple electrodes to patients with sensitive skin or who may be at a higher risk of severe skin breakdown, such as newborns or neonatal infants.

### SUMMARY

According to its major aspects and briefly recited, herein is disclosed an electrode system for neurological monitoring of patients with sensitive skin, such as newborn infants, the elderly, malnourished, and burn victims. The present electrode system includes an electrode, a housing, a lead wire, and a connector on the distal end of the wire. The electrode itself is made of a softer material than metal or epoxy, and the electrode is placed in electrical connection with the skin of the patient in a way to minimize physical connection, namely, via a conductive coating or gel between the electrode and the patient. The electrode and conductive gel are held within a recess in a foam housing. The housing is placed against the patient's head with the recessed side facing the head. A thin adhesive on the top surface of the wall that defines the recess and conductive gel is placed in the recess with the electrode. The housing thus both holds the electrode and conductive gel in the recess on the patient's side and cushions the patient's head against external forces. In addition, the electrode wires are thinner and more flexible, and they are attached to the electrode without welds or solder joints so as to minimize patient discomfort.

The softer conductive gel compared to the electrode extends the detection range of the electrode while limiting the electrode's direct contact with sensitive skin of the patient. The present electrode, which may be a “waffle-type” design that is more sensitive to electrical signals from the brain, is able to provide brain wave information with the intermediate presence of the conductive gel, and further allows for penetration and adhesion/confirmation with the conductive gel, reducing lateral movement and acquiring more accurate signals.

The present electrode is secured to the skin by a thin ring of film adhesive rather than a thicker adhesive “sticky pad” to limit skin damage on removal.

According to some further embodiments, an electrode system for sensitive skin is provided. An electrode system can comprise a sensitive skin electrode comprising a housing with wall defining a recess formed in said housing, an electrode in said recess, optionally, a conductive gel in said recess and in contact with said electrode, an electrical conductor having a first end and a second end, said first end of said electrical conductor being connected to said electrode in said recess, and a connector connected to said second end of said electrode wherein said electrode is made of silicone. The electrode system can further comprise a template structure, the template structure comprising a plurality of integrally connected template portions, and one or more electrode sites formed on the plurality of template portions configured to receive the sensitive skin electrode, wherein the one or more electrode sites are configured to contain a conductive gel.

Those skilled in the use and design of neurological electrodes will appreciate the many advantages of the present electrode system from a reading of the following Detailed Description in view of the accompanying drawings.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views. In the drawings:

FIG. 1 is an exploded view of the electrode system according to an aspect of the disclosure;

FIG. 2 is a side, cross-sectional, exploded view of a portion of the present electrode system, according to an aspect of the disclosure;

FIG. 3A is a side, cross-sectional view of a portion of the present electrode system prior to use, according to an aspect of the disclosure;

FIG. 3B is a side, cross-sectional view of a portion of the present electrode system in use, according to an aspect of the disclosure;

FIG. 4 is a top view of a set of electrode systems on a single release paper, according to an aspect of the disclosure;

FIG. 5 is a perspective view of an electrode template illustrating use with an electrode system, in accordance with an aspect of the present disclosure; and

FIG. 6 is an exploded view of a portion of an electrode template and a portion of an electrode system, in accordance with an aspect of the present disclosure.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Embodiments of the present neurological electrode system reduces patient discomfort and delays skin breakdown compared to other electrodes. In other respects, the present electrode system may be used in electro-encephalograph (EEG) or intra-operative monitoring (TOM) applications in the same manner as other neurological electrodes, that is, for stimulating a patient's brain or recording brain activity.

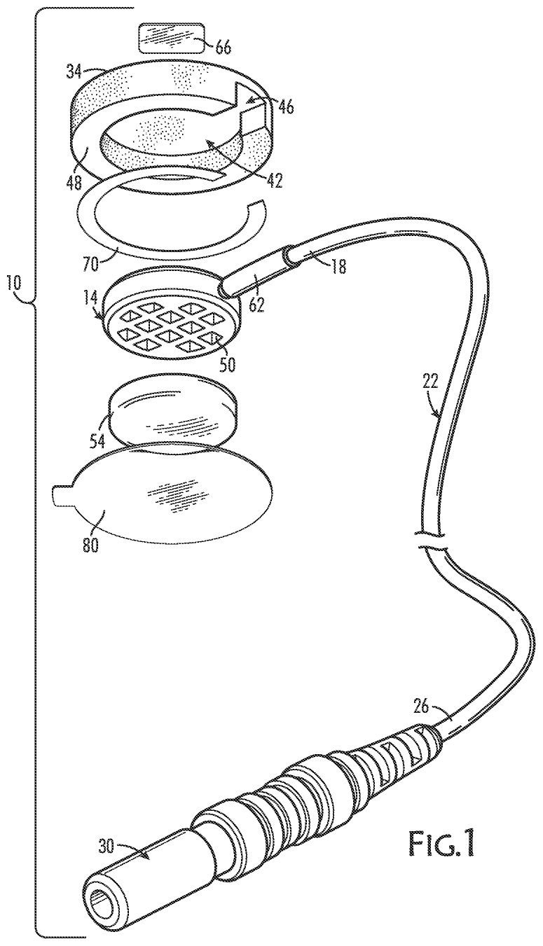

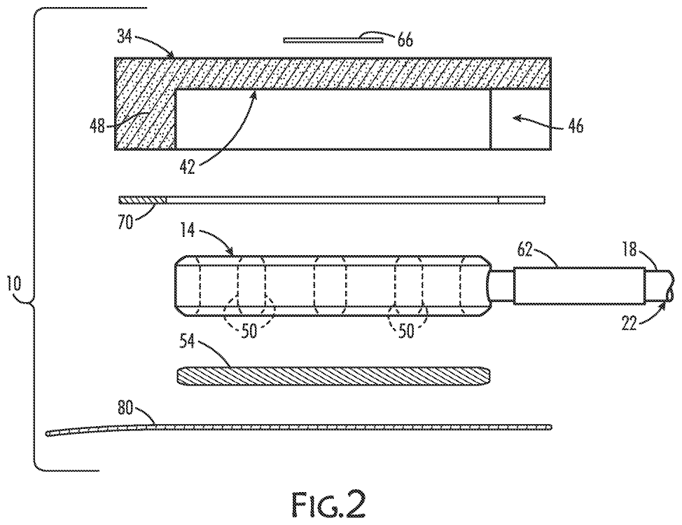

Referring now to FIGS. 1, 2, 3A, and 3B, the present electrode system 10 includes a housing 34 made of a resilient material such as a foamed plastic, for example, a closed-cell, cross-linked, nitrogen-impregnated, polyethylene foam. Housing 34 is therefore resilient and intended to serve as a cushion against external pressure such as when the head of the patient is resting against a pillow and the electrode is between the pillow and the patient's head. Housing 34 has a recess 42 formed therein and defined by a wall 48. The interior of recess 42 is dimensioned to receive an electrode 14, and may be dimensioned to hold electrode 14 securely. The inner recess also serves to protect the electrode 14 from damage, such as incidental damage in storing or in the placement thereof. Furthermore, the interior recess 42 may also reduce elemental damage, such as from water or corrosives on the electrode 14.

Electrode 14 may be made of a flexible material such as silicone rubber or acrylonitrile butadiene styrene (ABS) with an electrically conductive coating such as mixture of silver and silver chloride, and may be impregnated with a conductive material such as copper fibers or carbon fibers, such as graphite fibers. Electrode 14 may be formed to have passages 50 in it, and which may be through passages 50 to provide electrode 14 with more contact with a conductive gel 54 than if electrode did not have passages 50. Electrode 14 may be, for example, a WEBB electrode manufactured and sold by Rhythmlink, International, Inc.

Passages 50 in electrode 14 that receive an in-flow of conductive gel 54 help improve inter-surface contact between conductive gel 54 and electrode 14. These passages 50 work to increase the surface area contact with the conductive gel 54, thus amplifying the signals versus a flat electrode. Furthermore, the passages 50 allow for better adhesion to the conductive gel 54, and resist lateral shifting of the same.

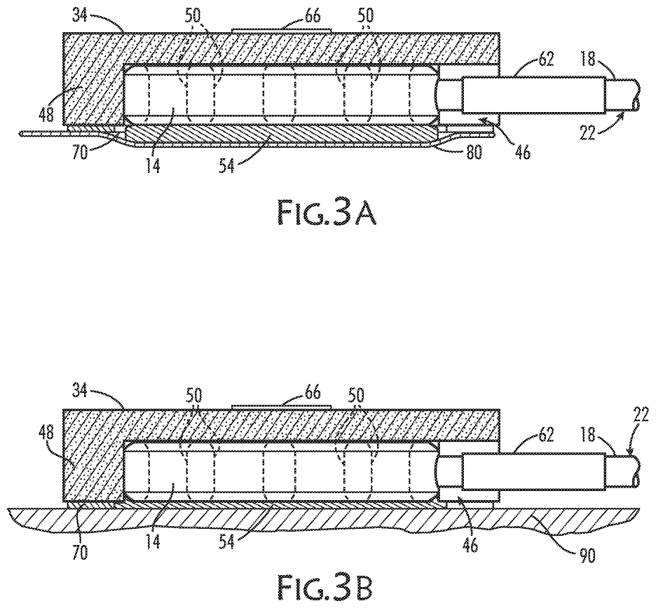

In some instances, a neonatal grade conductive gel may be used. Conductive gel 54, when in contact with electrode 14, extends the effective range of electrode 14 for detecting and/or sending electrical signals. Electrode 14 is not attached to the patient's skin 90 (best seen in FIG. 3B) and may not be in direct contact with the patient's skin 90. Electrode 14, however, can sense and transmit electrical signals so long as conductive gel 54 is in contact with skin 90 of the patient and gel 54 is in contact with electrode 14.

The surface 38 of wall 48 may include an adhesive ring 70 that will hold housing 34 to skin 90 of patient, where the adhesive of adhesive ring 70 may be a thin laminate adhesive. Adhesive ring 70 may be applied to surface 38 of wall 48 as a thin ring. A neonatal grade of skin adhesive may be used, such as a hydrogel-type adhesive to limit the extent and severity of skin injuries on removal. This gel may be comprised of other qualities to help preserve the integrity of the skin while being worn for long durations, such as lotions or nutrients.

Prior to use, a release paper 80 may be applied over wall 48 and recess 42 to protect adhesive ring 70, conductive gel 54, and electrode 14 prior to use. Release paper 80 is then peeled from electrode system 10 and the recess side of housing 34 with adhesive 70 being placed against the patient's skin 90.

The side of electrode 14 opposing the side with recess 42 may optionally carry an indicator of the position on the scalp of a patient designated for that electrode 14. The position designation corresponding to an indicator of a position on the scalp of a patient may be selected from those position designations in a standard set of position designations for the human head, which position designations are familiar to those of ordinary skill in the art of neurological monitoring.

Electrical conductors 22 have a first end 18 connected to electrode 14 in a manner that avoids hard surfaces such as welds or solder joints. For example, first end 18 of an electrical conductor 22 may be inserted into a sleeve 62 attached to electrode 14 and pass through an opening 46 in wall 48. A distal end 26 of electrical conductors 22 terminate in a connector 30 that is selected to be received directly by a neurological monitoring amplifier (not shown). Electrical conductors 22 used herein may also be made of a thinner gauge of metal, such as 18 gauge or higher, or of carbon. Electrical conductors 22 may comprise a wire having an electrically insulating coating of a more flexible electrically insulating material, such as one made of complex polymers like perfluoroalkoxy alkanes, fluoropolymers, copolymers, or other polymeric materials.

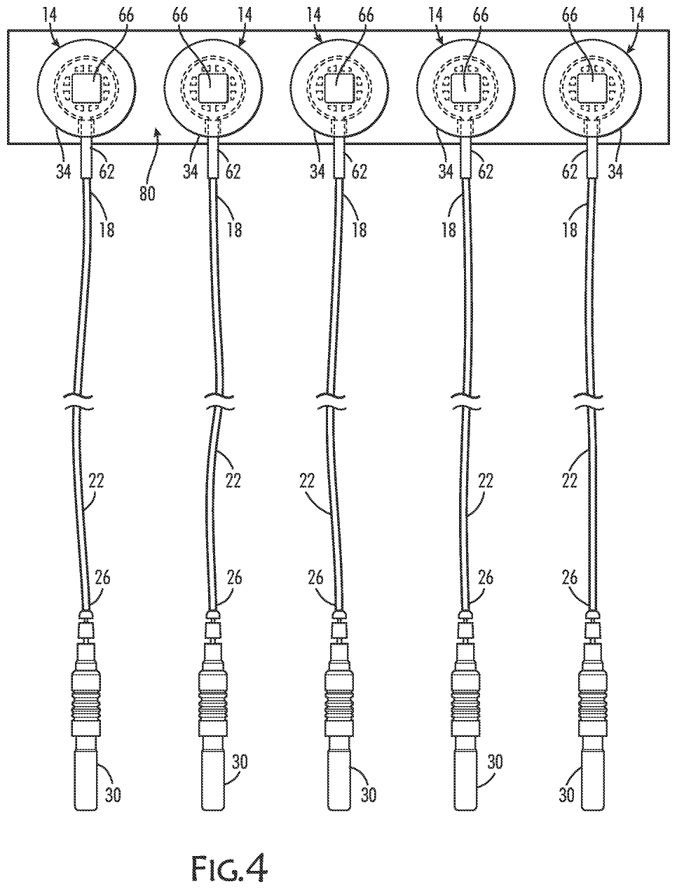

As seen in FIG. 4, a set of electrode systems 10 as described above may be needed and/or implemented for monitoring. Five electrode systems 10 are illustrated in FIG. 4, however, a different number may be used or required. A portion or all of the electrode systems 10 required for neurological monitoring or stimulating of an infant may be attached to a single, extended release paper 80 for convenience in a row and with the side of housing 34 opposite recess 42 facing down so that a position indicator 66 may be facing up toward the technician assigned to apply electrode systems 10.

According to some further aspects of the technology described herein, a template (i.e. electrode template) or sensitive skin template is provided for use with one or more electrodes or electrode systems. In some instances, a sensitive skin template can be formed as a single unit or through integrated or otherwise connected pieces, for example straps, wires, and template portions which can be joined or connected to form the sensitive skin template. The sensitive skin template, also referred to as template herein, can be made of any material which may have some degree of flexibility or deformability such that it varying patient head sizes. In some instances, some of the portions that form a sensitive skin template may be selectively expandable or tightened to fit a patient's head.

According to some embodiments, an electrode template for sensitive skin can be used with or as a part of an electrode system to apply multiple electrodes to patients with sensitive skin. In some aspects, an electrode template comprises a pre-gelled (i.e. gel-loaded for connectivity of electrodes), adhesive-lined, template with a plurality of potential electrode sites or connectivity sites. Accordingly, an electrode template can provide an adhesive or hydrogel for sensitive skin and further be implemented as a protective housing (formed, for example, from foam or other soft and/or flexible material) having a plurality of electrode sites or connectivity sites that are pre-filled or pre-loaded with gel which can be used in biosignal acquisition at each electrode site through connected electrode systems.

As will be appreciated, conventional systems which utilize standard electrodes on sensitive skin can cause issues with skin breakdown of a patient if for instance proper application and maintenance of electrodes isn't performed. Accordingly, as described further herein, embodiments of the present technology are directed to an electrode template which employs one or more electrode cavities that promotes floating of an electrode used in neurological monitoring by providing a pre-gelled or pre gel loaded cavity that an electrode can be inserted into but not be pressed to hard down on the contact point of a patient, e.g. the skin of a patient's head. Further, the re-application of gel in an EEG procedure can more easily be performed in that each electrode site (or electrode cavity, referred to also as connectivity site herein) has its own cavity with a dedicated port for inserting an electrode and gel. Additionally, as will be further appreciated, individual electrodes may be easily swapped at each electrode site or cavity.

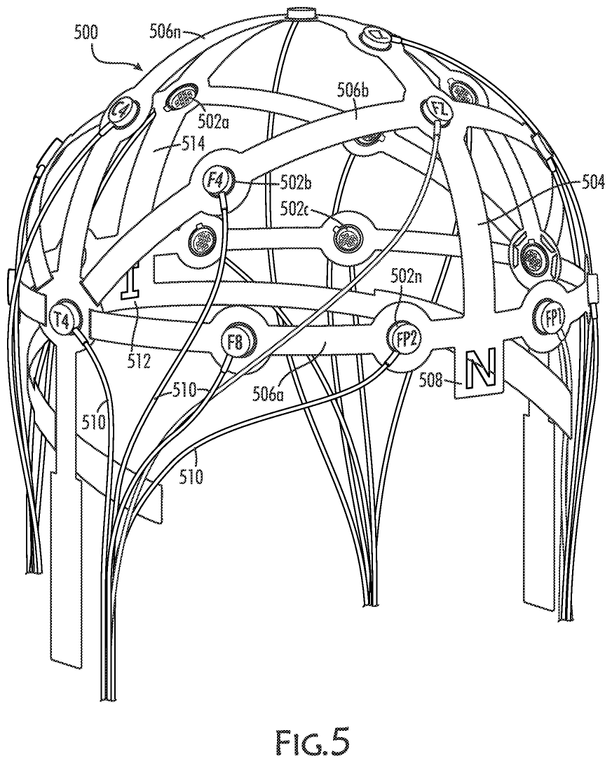

Referring now to FIG. 5, a template or template structure 500 for use in conjunction with one or more electrodes or electrode systems 510 is illustrated. Template structure 500 can comprise a plurality of electrode sites or connectivity sites 502a, 502b, 502c, 502n to which an electrode or electrode system 510 can be connected. As will be appreciated, template structure 500 can have as many or as few electrode or connectivity sites as required or desired. For example, in some embodiments the electrode or connectivity sites can be configured as a standardized 10-20 system in the context of an EEG exam. As illustrated, the electrode or connectivity sites 502a, 502b, 502c, 502n are connected by and at least in part formed from template structure 500, which may in some instances be a soft and/or flexible material. One or more electrodes or electrode systems 510 can be connected to each electrode site or connectivity site 502a, 502b, 502c, 502n. In some instances, the electrodes for connection with the template structure 500 can be standard cup electrodes, or for example WEBB electrodes, such as Ag/AgCl electrodes.

According to some aspects, the plurality of electrode sites or connectivity sites (e.g. 502a, 502b, 502c, 502n) are formed at least in part as a plurality of cutouts in a template structure 500. The locations of the electrode or connectivity sites may be predetermined, for example as a pattern prior to the forming of the template structure 500. Template structure 500 can further be made of a single piece of material and formed into a shape that fits a patient's head. In some instances, template structure 500 can be formed from a plurality of intersecting and/or attached and/or interwoven portions or strips. In some instances, the plurality of portions of the template structure 500 are integrally formed, i.e. formed from a single piece of material. For instance, template structure 500 can include at least one row portion 504 that can be integrally formed or connected with one or more column portions 506a, 506b, 506n, for example. According to some embodiments, all portions of a template structure 500 can be formed from one piece of material. In some other embodiments, discrete portions of template structure 500 can be formed separately and fastened or otherwise attached to form the complete template structure 500. Referring to template structure 500, row portion 504 can have a first end 508 (marked with an N to indicate Nasion) and a second end 512 (marked with an I to indicate Inion). The first end 508 and the second end 512 can be utilized to position the template structure 500 on a patient to correctly align the electrodes 510 and/or connectivity sites 502a, 502b, 502c, 502n.

According to some aspects, portions of template structure 500 may be lined or coated with an adhesive. For instance, template structure 500 can be lined with adhesive on any area of row portion(s) 504 and/or column portion(s) 506a, 506b, 506n, for example adhesive may line row portion 504 at 514. As will be appreciated portions of template structure 500 lined with adhesive can be configured to assist in the formation of template structure 500 or further for affixing template structure 500 to a patient or further to ensure electrodes 510 are connected to template structure at the determined electrode or connectivity sites. According to some further aspects, the electrode or connectivity sites 502a, 502b, 502c, 502n are configured at least in part as electrode openings or electrode cavities which can be sized to fit electrodes 510. Electrode openings or electrode cavities may be loaded with a gel (e.g. pre-loaded) for neurological monitoring prior to attachment of electrodes 510. According to some embodiments, pre-loaded electrode cavities can enable or promote electrode floating, by providing a pre-gelled cavity that an electrode (e.g. electrodes 510) can be inserted into but not be pressed down too hard on a patient's skin (e.g. skin of a patient's head) or contact point.

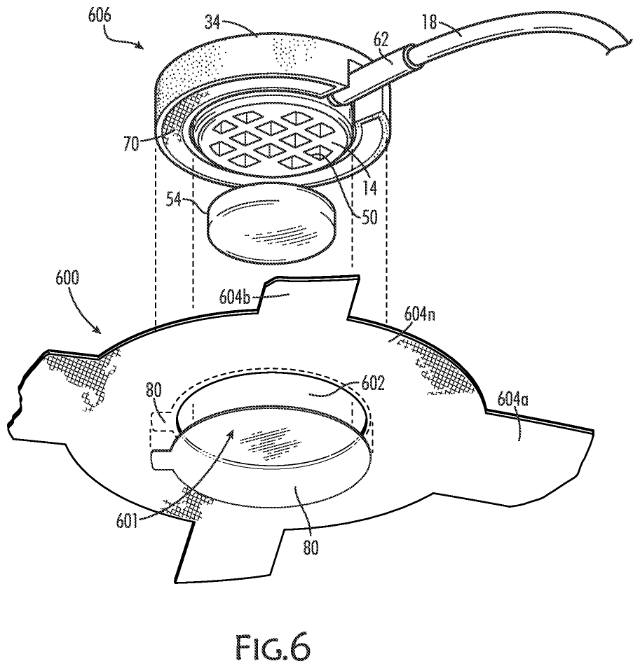

Referring to FIG. 6, an exploded view of an electrode system for sensitive skin comprising, among other things, an electrode 606 and a template structure 600, where the template structure is configured for use with sensitive skin. In some instances, embodiments of the technology described herein may utilize a standardized electrode in conjunction with a template structure 600, conductive gel 54, and release paper 80. In some other instances, an electrode 606 can be used which includes, amongst other things, housing 34, electrode 14 which can include passages 50. Additionally, electrode 606 can include an adhesive 70 which is configured to attach electrode 606 to template structure 600. Template structure 600 can be formed from one or more portions 604a, 604b, 604n, and can be formed from a single piece of material. As previously described any of portions 604a, 604b, 604n can have an adhesive coating thereon (such as a sensitive skin adhesive) for attaching template structure 500, 600 to a patient's head. Template structure 600 can be formed from one or more materials including, but not limited to, foam, polymers, woven materials, synthetic, non-woven, laminated, and/or fibrous material. Generally, (and with reference to FIG. 5), template structure 600 can have one or more or a plurality of electrode or connectivity sites, referred to generally as 601 (e.g. 502a, 502b, 502c, 502n of FIG. 5). Template structure 600 can further comprise an adhesive coating 81 to attach release paper 80 to one side of the template structure 600 to contain a loaded conductive gel 54. In some instances, the template structure 600 and release paper 80 can form a well or cavity 602 for the pre-loading of conductive gel 54 (e.g. hydrogel for sensitive skin). According to various embodiments, template structure 500, 600 can be pre-loaded with gel, and having an adhesive lining on the side of the template structure that is in contact with a patient's head. Prior to use release paper 80 is then peeled from template structure 600 and the recess side of housing 34 with adhesive 70, and electrode 14 being placed against the patient's skin, having a layer of conductive gel 54 there between that is configured to the passages to reduce lateral movement.

The template structure may further be coated in an antibacterial layer, thus resisting containment's. In other aspects, the template structure may be coated in friction reducing material, such as Teflon, or other material that reduces the amount of friction felt by the patient. In other aspects the template structure may be comprised of elastic material allowing for compression and a fit that allows flexibility for jaw or facial musculature.

Those skilled in the art of neurological electrodes will appreciate that many modifications and substitutions may be made in the electrode system as herein described without departing from the spirit and scope of the present disclosure.

That which is claimed:

1. An electrode system for sensitive skin, comprising:
(a) a sensitive skin electrode, comprising:
(i) a housing with a wall defining a recess formed in said housing;
(ii) an electrode in said recess;
(iii) optionally, a conductive gel in said recess and in contact with said electrode;
(iv) an electrical conductor having a first end and a second end, said first end of said electrical conductor being connected to said electrode in said recess;
(v) a connector connected to said second end of said electrode wherein said electrode is made of silicone; and
(b) a template structure, comprising:
(i) a plurality of integrally connected template portions;
(ii) one or more electrode sites formed on the plurality of template portions configured to receive the sensitive skin electrode, wherein the one or more electrode sites are configured to contain a conductive gel.

2. The electrode system of claim 1, further comprising the one or more electrode sites formed by a release paper adhered to one side of the template structure opposing the sensitive skin electrode receiving side of the template structure.

3. The electrode system of claim 1, further comprising one or more areas of the plurality of template portions comprising an adhesive coating.

4. The electrode system of claim 1, further comprising the template structure comprising a plurality of electrode sites, the plurality of electrode sites corresponding to a 10-20 configuration.

5. The electrode system of claim 4, further comprising the plurality of electrode sites loaded with the conductive gel prior to attachment of the sensitive skin electrode.

6. The electrode system of claim 1, further comprising the template structure being formed from a foam.

7. The electrode system of claim 1, further comprising an opening being formed in said wall for said electrical conductor to pass therethrough.

8. The electrode system of claim 1, further comprising said electrode system comprising:
(a) an adhesive on said wall; and
(b) a release paper on said adhesive.

9. The electrode system of claim 1, further comprising said housing is made of foamed plastic.

10. The electrode system of claim 1, further comprising said electrode having passages formed therein, and said conductive gel penetrating into said passages when said electrode and said conductive gel are in said recess.

11. The electrode system of claim 1, further comprising said electrical conductor being comprised of carbon fibers.

12. The electrode system of claim 1, further comprising said electrode being comprised of silicone rubber coated with conductive material.

13. The electrode system of claim 1, further comprising said electrode being comprised of silicone rubber filled with conducting fibers.

14. The electrode system of claim 1, further comprising said electrode being comprised of acrylonitrile butadiene styrene with an electrically conductive coating.

15. The electrode system of claim 1, further comprising said electrode being comprised of passages to allow the conductive gel to adhere and reduce lateral movement.

16. The electrode system of claim 1, wherein said electrode is comprised of acrylonitrile butadiene styrene with a conductive coating made of a mixture of silver and silver-chloride.

17. The electrode system of claim 1, further comprising a sleeve, and said sleeve is operable to connect said electrical conductor to said electrode into said sleeve.

18. The electrode system of claim 1, further comprising said housing being comprised of polyethylene foam.

19. The electrode system of claim 1, further comprising said conductive gel being a neonatal conductive gel.

20. The electrode system of claim 1, further comprising said housing carrying an electrode position designation.

21. The electrode system of claim 1, further comprising the template structure lined with adhesive, the adhesive being designed for sensitive skin.
